(19) United States
(12) Patent Application Publication
(10) Pub. No.: US 2024/0360159 A1
(43) Pub. Date: Oct. 31, 2024

(54) NOVEL APPLICATIONS FOR OXAZABOROLIDINES AND NEW METHODS OF MANUFACTURE THEREOF

(71) Applicant: Pathway Intermediates Limited, Shrewsbury Shropshire (GB)

(72) Inventors: David Garnett, Shrewsbury Shropshire (GB); Lloyd Cooper, Shrewsbury Shropshire (GB)

(73) Assignee: Pathway Intermediates Limited, Shrewsbury Shropshire (GB)

(21) Appl. No.: 18/291,631

(22) PCT Filed: Jul. 8, 2022
(86) PCT No.: PCT/EP2022/069142

§ 371 Date: Jan. 24, 2024

(30) Foreign Application Priority Data
Aug. 3, 2021 (GB) 2111203.2

(51) Int. Cl. C07F 5/02; A61K 31/69; A61P 43/00

(52) U.S. Cl. CPC C07F 5/027; A61K 31/69; A61P 43/00

(57) ABSTRACT

Processes for producing an oxazaborolidines and their use in animal feeds. One process reacts an amino alcohol with a boronic acid with azeotropic removal of water to provide a mixture of the starting materials and an oxazaborolidine end product; and purifying the oxazaborolidine end product. One purification step dissolves a bicarboxylic acid in water and mixes this solution with the end product to precipitate out the starting material as a salt and produce an oxazaborolidine product filtrate. An alternative process reacts amino alcohol with trihydridoboron (BH₃) in an organic solvent; and immediately alkylates the unalkylated intermediate compound produced using Alkyl Lithium to provide the oxazaborolidine end product.

### FIELD OF THE INVENTION

The present invention relates to improved methods for preparing oxazaborolidines and to novel applications for said oxazaborolidines.

### BACKGROUND

Quorum sensing is the ability to detect and respond to cell population density by regulation of gene expression. It is this chemical signalling between bacteria that enables them to communicate. Bacteria export chemical signalling molecules into their environment (Bassler, B & Lossick, R [2006]. Bacterially Speaking, Cell, pp 237-246) and the information supplied by these molecules is critical for synchronizing their activity. This chemical communication by bacteria involves producing, releasing, detecting and responding to the accumulation of small hormone-like molecules, called ‘autoinducers’. The cell-cell communication mechanism known as quorum sensing allows bacteria to monitor their environment for other bacteria, and to alter behaviour on a population-wide scale in response to changes in the number and/or species present in a community (Waters, C. & Bassler, B. I., [2001]. The Languages of Bacteria. Genes & Development. Volume 15, pp 1469-1480). For example, quorum sensing enables bacteria to restrict the expression of specific genes to the high cell densities at which the resulting phenotypes will be most beneficial. Many species of bacteria use quorum sensing to coordinate gene expression according to the density of their local population. Quorum sensing has been found to be an integral regulatory component of cellular processes such as bioluminescence production, virulence gene expression, biofilm formation, cell division, motility, metabolism and recombinant protein production.

Bacteria require three characteristics in order to use quorum sensing. The bacteria must 1) secrete a signalling molecule (the autoinducer), 2) detect the change in concentration of the signalling molecule, and 3) regulate gene expression in response. Different types of bacterial have different autoinducers. Acylated homoserine lactones (AHLs) are used by gram-negative bacteria. These molecules are synthesised by LuxI, and are detected by the LuxR protein. The LuxR-AHL complex act as a transcription factor to regulate gene expression (Schauder, S. & Bassler, B., The languages of bacteria. Genes and Development, Vol 15, pp 1469-1480). Gram-positive bacteria secrete modified oligopeptides as autoinducers. These oligopeptides are detected by membrane-bound, two-component adaptive response proteins. Binding of the oligopeptide autoinducer to these response proteins at a critical concentration triggers an intracellular phosphorylation cascade that cumulates in the phosphorylation, and subsequent activation, of the cognate response regulator (Ng, W L. & Bassler, B L., [2009] Bacterial Quorum-Sensing Network Architectures. Annu Rev Genet, Vol 43, pp 197-222). Upon activation, the response regulator acts as a transcription factor, resulting in modulation of gene expression (Kleerebezem, M et al., Quorum Sensing by peptide pheromones and two-component signal-transduction systems in gram-positive bacteria. Molecular Microbiology, Vol. 24, pp. 895-904).

A third type of autoinducer is autoinducer 2 (AI-2), a furanosyl borate diester and one of the only few known biomolecules incorporating boron. It is a signal molecule produced by the protein LuxS, an enzyme found in many bacterial species. AI-2 is thought to be responsible for switching on a large number of metabolic and catabolic pathways. Critically, it may be used by both gram-positive and gram-negative bacteria. Therefore, it is considered to be an important signal for inter-species communication and as such may be exploited in alternative strategies to antibiotic growth promotors.

The ongoing emergence of antibiotic-resistant bacterial strains means alternative therapeutic strategies are required. The ability of autoinducers to influence population-wide activity of bacteria, such as their growth and pathogenicity, indicates that they are feasible candidates for use as an alternative to antibiotic treatments. Quorum sensing can be influenced through the use of external autoinducers to inhibit the growth of pathogenic bacteria while promoting, or maintaining, the growth of beneficial bacteria. This variation in effect is possible due to (i) the variable threshold concentrations between bacteria, and (ii) the differing pathways induced by the same autoinducer across different populations of bacteria.

Oxazaborolidines have been used to control biofilm production because they are structurally similar to autoinducer-2 (AI-2). Oxazaborolidines are five-membered heterocyclic boron compounds containing both oxygen and nitrogen atoms and which can be synthesized to have a chemical structure that resembles that of AI-2 (Aharoni, R. et al., [2008]. Oxazaborolidine derivatives inducing autoinducer-2 signal transduction in Vibrio harveyi. Bioorganic & Medicinal Chemistry, Vol. 16, pp. 1596-1604). This structural similarity suggests that the oxazaborolidines may provide a similar biological effect and, indeed, several oxazaborolidines have been found to have physiological/metabolic and enzymatic effects on bacterial adhesion to the substrate. Oxazaborolidines have also been found to have modulatory effects on anti-enzymatic bacterial activity, act as anti-bacterial agents, and, pertinent to this application, have been shown to influence quorum sensing between bacteria, leading to a decrease in communication and a resultant damage to the integrity of bacterial biofilms.

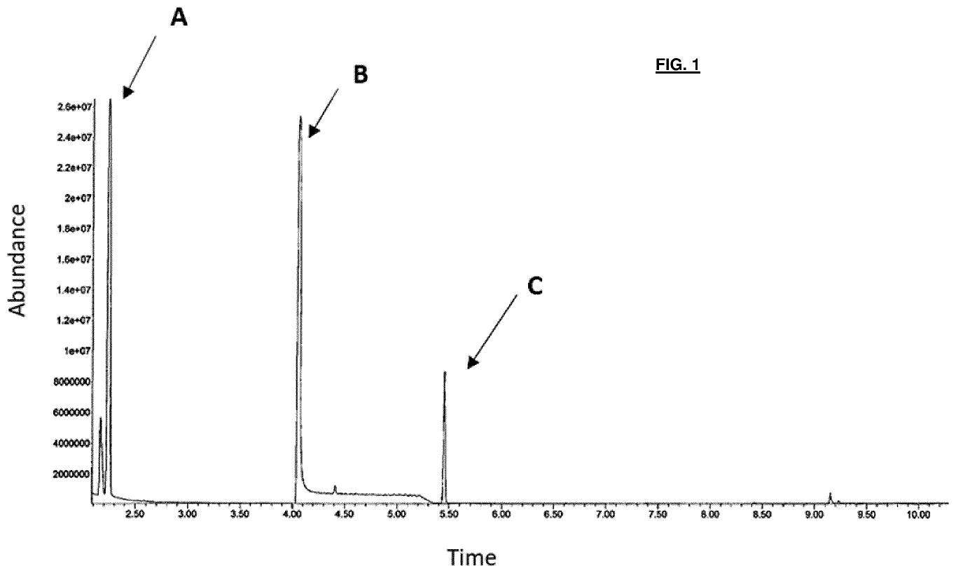

WO 2005/021559 A2 (Yissum Research Development Company of the Hebrew University of Jerusalem) discloses oxazaborolidines as bacterial effectors for modulating at least one bacterial-related parameter selected from the adhesion of the bacteria to its substrate; enzymatic activity of the bacterial enzymes; viability of the bacteria; effect on quorum sensing and biofilm formation by the bacteria. This document describes a number of different oxazaborolidine structures but fails to disclose a process that produces a pure oxazaborolidine compound. FIG. 1 of the accompanying drawings illustrates Gas Chromatography Mass Spectrometry (GCMS) spectra for the end product produced by the method disclosed in WO 2005/021559 A2. Three significant peaks are shown, the starting materials (A) butylboronic acid and (B) (−) ephedrine and (C) oxazaborolidine, with the end product (C) making up only around 10% of the end product.

It is the aim of the present invention to provide improved processes for the production of oxazaborolidines that overcome, or at least alleviate, the abovementioned drawbacks.

It is a further aim of the present invention to provide an alternative synthesis routes for production of novel oxazaborolidine autoinducers.

It is yet a further aim of the present invention to provide new uses for such oxazaborolidine derivatives.

### SUMMARY OF THE INVENTION

According to a first aspect of the present invention there is provided a process for producing an oxazaborolidine, the process comprising the steps of:
(a) reacting an amino alcohol with a boronic acid with the azeotropic removal of water to provide a mixture of the starting materials and an oxazaborolidine end product; and
(b) purifying the oxazaborolidine end product to at least 75% purity.

Preferably, the amino alcohol is (−) ephedrine, (−) norpseudoephedrine ethanolamine, ethanolamine or N-benzylethanolamine or a diol amine containing an aromatic moiety.

Preferably, step (a) is carried out under reflux for at least 1 hour, preferably in an organic solvent. Preferably, the organic solvent is Toluene. Preferably, the water produced in step (a) is removed and the organic solvent evaporated off, followed by vacuum distillation to provide impure end product of step (a).

In one embodiment of the present invention, the process includes a purification step (b) comprising dissolving a bicarboxylic acid in water and mixing this solution with the product of step (a) to precipitate out the starting material as a salt and produce an oxazaborolidine product filtrate.

Preferably, an equimolar amount of bicarboxylic acid is used vs the starting material impurity.

The bicarboxylic acid may be oxalic, adipic or citric acid depending on reaction conditions and desired solvent systems. More preferably the acid is oxalic acid. The oxalic acid in water in step (b) is preferably heated to at least 40° C., more preferably 50° C. More preferably, the precipitate is filtered off and the product filtrate is dried by evaporation. Preferably the purity of the end product is greater than 90%, preferably greater than 98%, especially 100%. The yield is preferably at least 60%.

Preferably, the boronic acid is a butyl- or phenylboronic acid.

In an alternative embodiment of the present invention, the process includes a purification step (b) comprising a vacuum distillation step to provide substantially pure oxazaborolidine, without addition of a bicarboxylic acid.

In this embodiment, any organic solvent with the impure oxazaborolidine prepared in step (a) is removed and the residue mixed with a solvent, preferably a volatile solvent such as an organohalide solvent, for example dichloromethane (DCM) and subjected to vacuum distillation, more preferably being at a high vacuum and temperature, preferably at least 0.5 mbar and at least 100° C., to provide a substantially pure oxazaborolidine as the distillate. More preferably still, Kugelrohr vacuum distillation is used to obtain the pure product. It is preferable for the amino alcohol in step (a) to be ethanolamine or N-benzyl ethanolamine and the boronic acid to be phenylboronic acid.

A second aspect of the present invention provides an alternative process for producing an oxazaborolidine, the process comprising the steps of:
(a) reacting an amino alcohol with trihydridoboron (BH₃) in an organic solvent; and
(b) immediately alkylating an unalkylated intermediate compound produced by step (a) using Alkyl Lithium to provide the oxazaborolidine end product.

Preferably, the organic solvent used in step (a) is DMS or THF, more preferably THF. Reaction (a) is preferably carried out at a temperature of at least 60° C., preferably 66° C.

The alkyl lithium in step (b) is preferably Butyl- or Phenyl Lithium. Preferably, step (b) is carried out at a temperature of at least −50° C., preferably −78° C.

Preferably the purity of the end product is greater than 90%, preferably greater than 98%, especially 100%. The yield is preferably at least 60%, especially at least 80%.

Preferably, the process according to the first or second aspect of the present invention prepares an oxazaborolidine having the general formula (I) below:

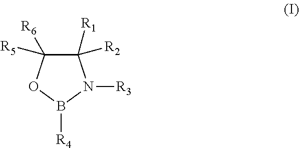

wherein
R1, R2, and R3 are each independently selected from hydrogen, C₁-C₈ alkyl, C₂-C₈ alkenyl, aryl, and C₃-C₇ cycloalkyl or one of R1 and R2 together with R3 form a substituted or unsubstituted aromatic ring, a substituted or unsubstituted cycloalkyl ring, or a substituted or unsubstituted heterocyclic ring, fused to the oxazaborolidine ring; R4 is selected from hydrogen, hydroxyl, C₁-C₈ alkyl, C₂-C₈ alkenyl, aryl, and C₃-C₇ cycloalkyl;
R5 and R6 are each independently selected from hydrogen, C₁-C₈ alkyl, C₂-C₈ alkenyl, aryl, and C₃-C₇ cycloalkyl.

Preferably, one of R1 and R2 is an alkyl group having 1-4 C atoms, most preferably methyl, with the other being hydrogen. Preferably, R3 is an alkyl group having 1-4 C atoms, most preferably a methyl group, with the other being hydrogen. Alternatively, one of R1 and R2 together with R3 form a substituted or unsubstituted heterocyclic ring fused to the oxazaborolidine ring to provide a pyrrolidine.

More preferably, R4 is selected from the group consisting of an alkyl group having 1-4 C atoms, a hydrogen or a phenyl group, especially methyl, butyl or a phenyl group.

In a preferred embodiment at least one or both R5 and R6 is a phenyl group, with the other being hydrogen.

It is to be appreciated that the end product of the processes according to the first and second aspects of the present invention is substantially free of any starting material, representing a significant improvement over prior art processes.

A third aspect of the present invention provides a feed additive for an animal or bird, especially poultry, wherein the feed additive comprises an oxazaborolidine having the general formula (I) as defined above.

The feed additive is preferably added to a basal diet in a substantially pure form. Preferably, the additive is provided in a dose of 100-300 g per tonne of animal feed, preferably, 125-275 g/tonne, more preferably 150 g.

It is preferable to supplement an animal feed with 0.05-0.2% of the oxazaborolidine, more preferably, 0.05-0.1%. This generally relates to a percentage per tonne of animal feed.

The feed additive preferably comprises the oxazaborolidine, 2-butyl-3,4 dimethyl-5-phenyl 1,3,2-oxazaborolidine.

The feed additive improves gut flora and growth performance of the bird.

Preferred oxazaborolidines for use in the third aspect of the present invention have the general formula (I) above, especially being one of the following oxazaborolidines:

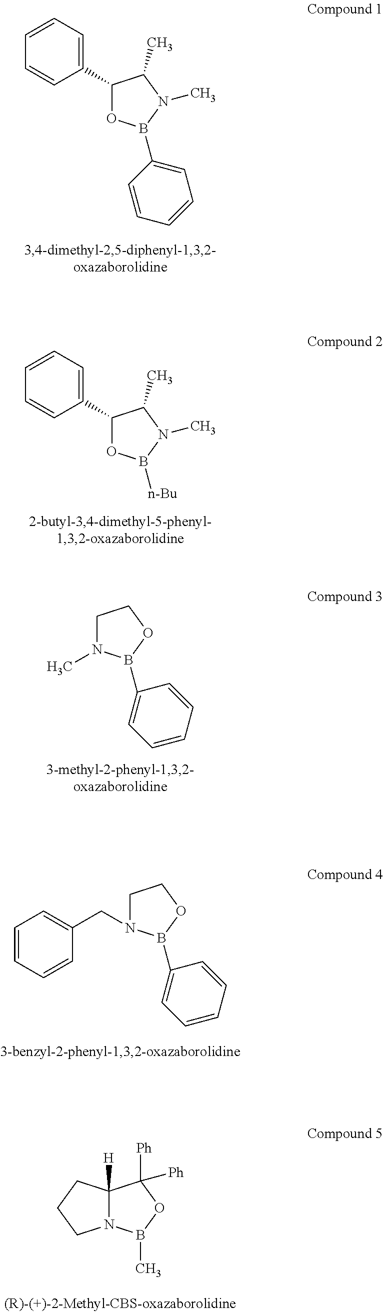

### BRIEF DESCRIPTION

The invention will now be illustrated with reference to the following drawings and examples in which:

FIG. 1 is a GCMS spectra for impure oxazaborolidine prior to purification by a process according to the present invention;

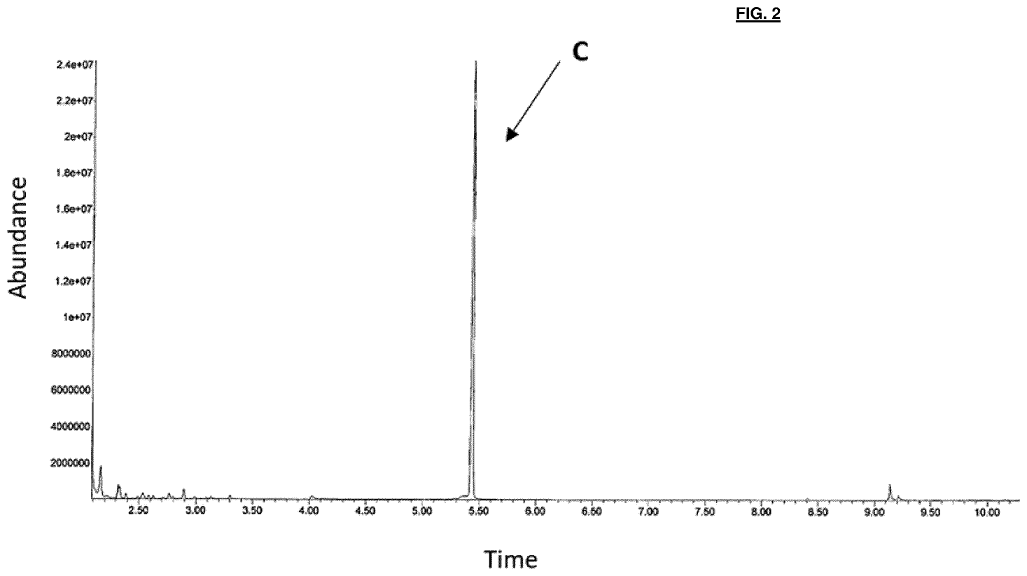

FIG. 2 is a GCMS spectra for pure 2-butyl-3,4-dimethyl-5-phenyl-1,3,2-oxazaborolidine produced by a process according to one embodiment of the present invention;

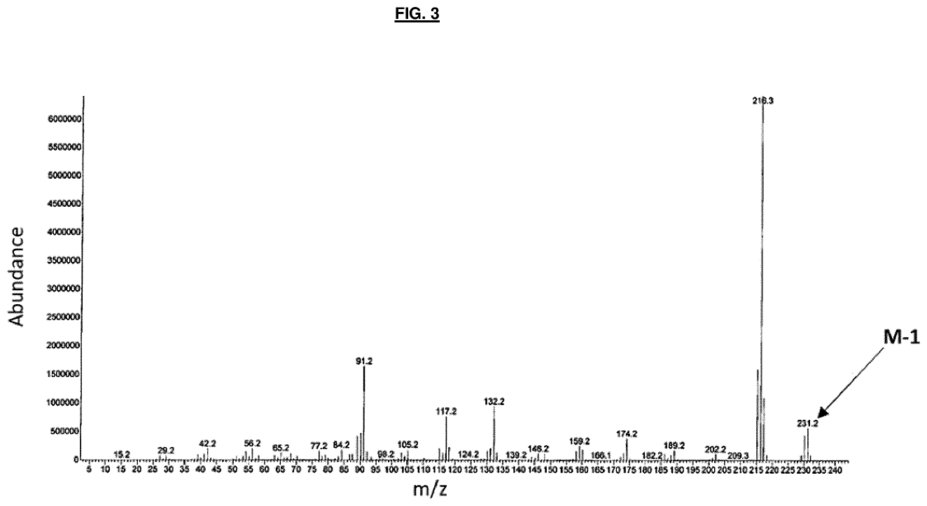

FIG. 3 is a GCMS fragmentation pattern for 2-butyl-3,4-dimethyl-5-phenyl-1,3,2-oxazaborolidine;

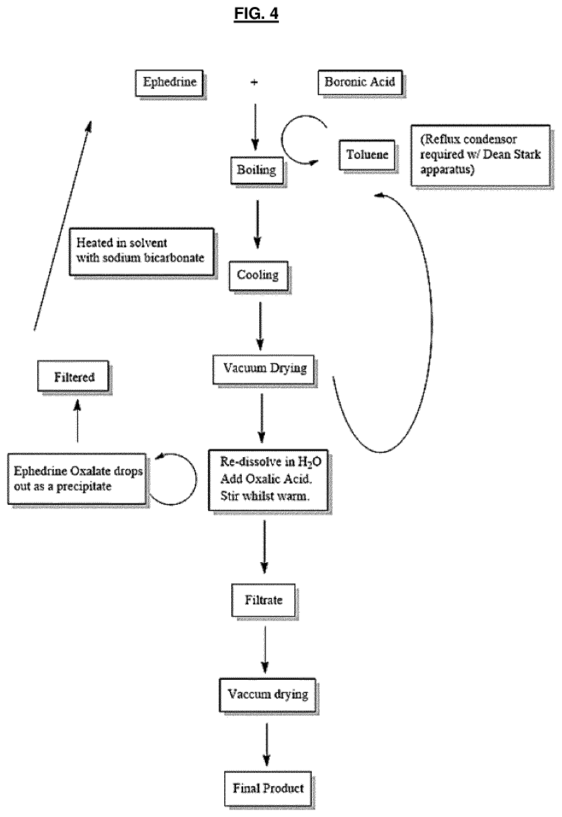

FIG. 4 is a reaction scheme for a scaled-up process for producing oxazaborolidines according to one embodiment of the present invention;

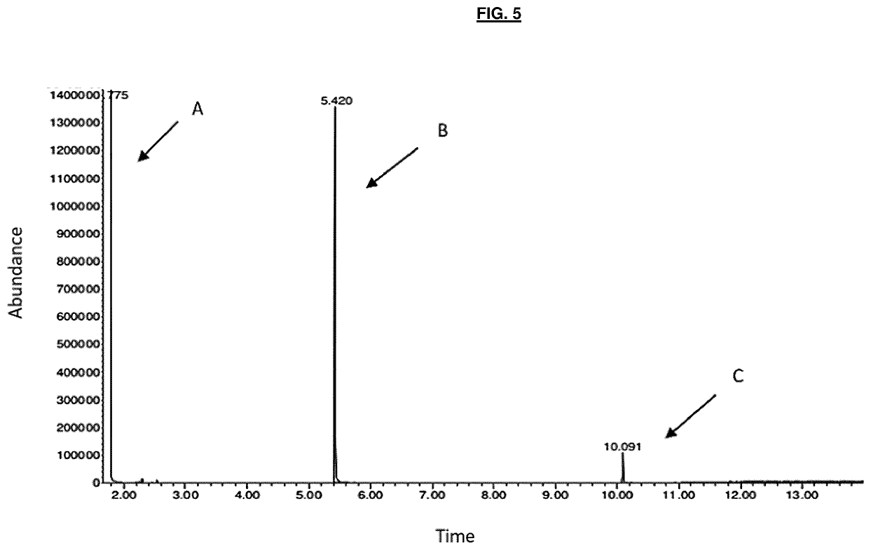

FIG. 5 is a GCMS spectra for 3-methyl-2-phenyl-1,3,2-oxazaborolidine produced by a process according to another embodiment of the present invention;

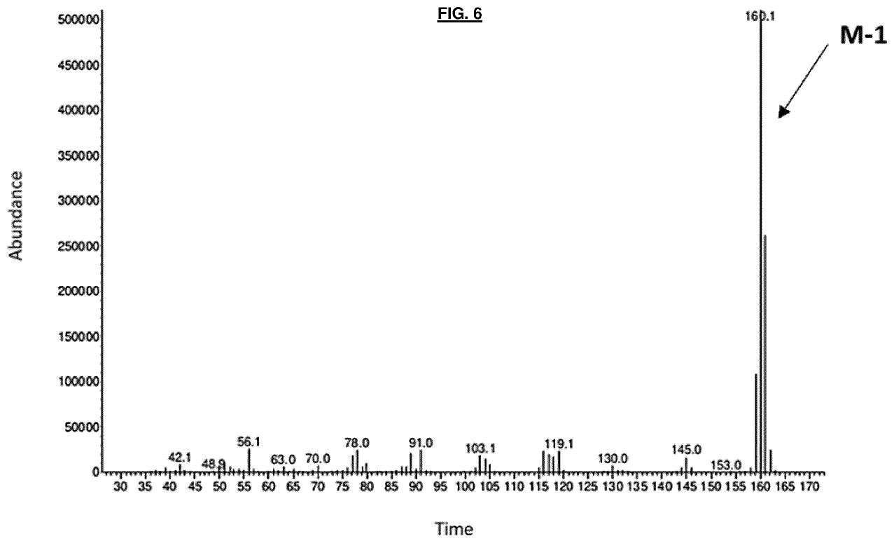

FIG. 6 is a GCMS fragmentation pattern for 3-methyl-2-phenyl-1,3,2-oxazaborolidine;

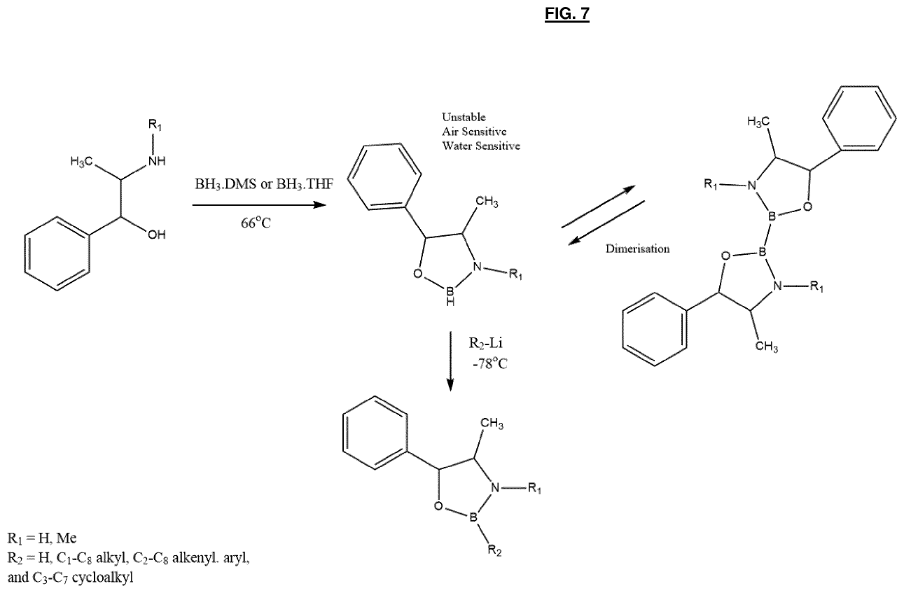

FIG. 7 is a reaction scheme for producing oxazaborolidines according to yet another embodiment of the present invention; and

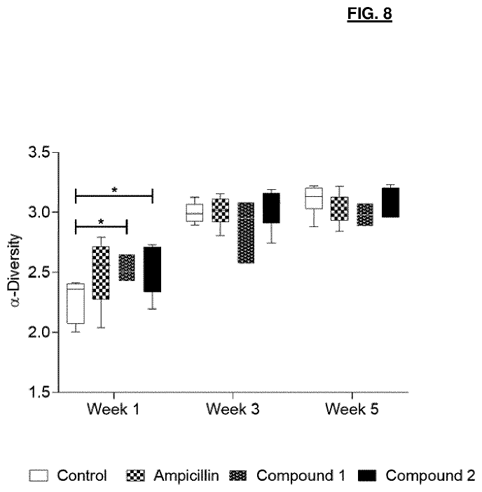

FIG. 8 is a graph detailing the changes in bacterial α-diversity following dietary supplementation of broiler chickens fed with a dietary supplement of oxazaborolidines prepared according to Example 1 of the invention.

### DETAILED DESCRIPTION

The present invention provides improved methods for preparing oxazaborolidines and new uses for these compounds.

Oxazaborolidines are known to be potentially useful as autoinducers but prior hereto it has not been possible to satisfactorily produce sufficiently pure compounds for different applications, particularly in large quantities. The present invention provides novel processes for producing the purified compounds and additionally, their novel application as an animal and bird feed additive, particularly poultry.

### Example 1: Preparation of Pure Oxazaborolidines According to One Embodiment of the Present Invention

Oxazaborolidines according to the general formula (I) were synthesized by reacting an amino alcohol. Specific examples include, but are not limited to; (−) ephedrine, (−) norpseudoephedrine, ethanolamine, N-Benzyl ethanolamine, or a diol amine containing an aromatic moiety with an appropriate boronic acid, with the azeotropic removal of water, as set out below:

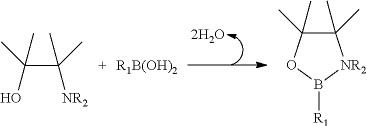

The reaction mixture was refluxed for around 2-3 hours, in an organic solvent, preferably Toluene, and the water removed. The compound was then isolated by evaporating off the organic solvent to leave the residue. This was vacuum distilled to obtain the desired compound as a mixture of starting materials and product, as demonstrated in FIG. 1, where (A) corresponds to butylboronic acid, (B) is ephedrine and (C) is 2-butyl-3,4-dimethyl-5-phenyl-1,3,2-oxazaborolidine. This mixture also corresponds to the end product obtained in WO 2005/021559 A2.

Crucially, the process according to this embodiment of the present invention incorporates a further purification step to provide a substantially pure oxazaborolidine product. This was achieved by dissolving oxalic acid in water and then, preferably heating the mixture to at least 40° C., preferably to 50° C. An equimolar amount of oxalic acid (vs starting material impurity) was made in the solution and then the mixture of product and starting materials obtained in step (a) was added to the solution. The oxalic acid reacts with the (−) ephedrine to produce an ephedrine oxalate by-product which precipitates out of solution. This was filtered off to leave the oxazaborolidine product filtrate. The filtrate was dried by evaporation to yield a fine, white powder. The Gas Chromatography Mass Spectrometry (GCMS) spectra for the end product produced by this method is shown in FIG. 2. It is clear that only the oxazaborolidine (C) is present, demonstrating a pure end product. The yield was approximately 60%. FIG. 3 shows the fragmentation pattern for the compound, again demonstrating that the correct pure compound is produced.

The ability of this process to recover the waste starting material also provides an additional benefit over the prior art process. The (−) ephedrine starting material is a precursor for a number of controlled substances, including amphetamines, and therefore it is imperative that none of this material remains in the end product.

This process is particularly suitable when using butyl or phenylboronic acids as one of the starting materials.

FIG. 4 of the accompanying drawings illustrates a scaled-up version of this process in a large QVF™ (borosilicate) glass reactor.

### Example 2: Preparation of Pure Oxazaborolidine Compounds According to Another Embodiment of the Present Invention

This example follows the same general synthesis method as Example 1, but does not require the oxalic acid purification step, instead utilizing an alternative purification step. This produces oxazaborolidines according to the general formula (1).

An amino alcohol, typically ethanolamine or N-benzyl ethanolamine, was again reacted with a boronic acid, specifically phenylboronic acid. The mixture was refluxed for 1 hour in an organic solvent, preferably toluene. The compound was then isolated by evaporating off the organic solvent to leave an oil-like residue.

Crucially, the process according to the present invention again incorporates a further purification step that provides a substantially pure oxazaborolidine product. This was achieved by removing the volatiles to give a yellow oil which forms a glass on cooling. This residue was transferred to a Kugelrohr bulb via heat and a suitable solvent, preferably DCM. DCM was removed under vacuum and the residue was pumped under high vacuum and high temperature, preferably <0.5 mbar and preferably at 100° C. The product; a pale-yellow oil is distilled across, leaving a small quantity of pink-brown residue containing impurities.

The Gas Chromatography Mass Spectrometry (GCMS) spectra in FIG. 5 shows an example end product produced by this method, in this instance Compound 3 (3-methyl-2-phenyl-1,3,2-oxazaborolidine (B). Peaks correspond to solvent run-off (acetone) (A); 3 (3-methyl-2-phenyl-1,3,2-oxazaborolidine (B); and triphenylboroxine (C). The triphenylboroxine peak occurs as the oxazaborolidine passed through the column and is subjected to high temperatures. FIG. 6 displays the fragmentation pattern for (C), showing the M-1 peak with a m/z ratio of 160.1.

### Example 3: Preparation of Pure Oxazaborolidines According to a Third Embodiment of the Present Invention

An alternative, more versatile process for preparing oxazaborolidines according to the present invention is set out below. However, it has the drawback that is uses butyl lithium which is a volatile component.

The general reaction scheme is illustrated in FIG. 7 of the accompanying drawings.

The oxazaborolidine is synthesised by the reaction of an appropriate amino alcohol, namely (−) ephedrine and (−) norpseudoephedrine, together with trihydridoboron or borane (BH₃) in DMS, or more preferably THF.

The first step is driven by loss of Hydrogen and is irreversible. The unalkylated intermediate compound is then immediately alkylated using Phenyl Lithium or Butyl Lithium to give the alkylated compound which is stable.

This alternative approach gives a much higher yield than the process of Example 1, 85% yield vs 60%.

### Example 4: Investigation into the Use of Pure Oxazaborolidine (R)-(+)-2-Methyl-CBS-oxazaborolidine (Compound 5) as a Growth Promotor in Poultry

The pure oxazaborolidine compound 5 (R)-(+)-2-Methyl-CBS-oxazaborolidine prepared according to Example 1 was investigated to assess its potential as a growth promoter in poultry.

240 birds, 1-day old broiler (Ross 308; mixed sex) were treated over 32 days. Two dietary groups were given six replicates per diet; 40 birds per replicate. The two treatments consisted of (1) a control basal diet and (2) feed supplemented with a feed additive according to an embodiment of the present invention (0.055% (R)-(+)-2-Methyl-CBS-oxazaborolidine).

The feeding programme followed a typical feeding program on a farm (3 phases: starter; grower; finisher) with the feed additives added to the basal diet with top dressing.

The following measurements were taken:

Growth Performance: Body weight (BW); Weight gain (Average Daily Gain-ADG); Feed intake (Average Daily Feed Intake—ADFI); FCR (food conversion ratio-feed input divided by feed output).

The effect of the additive on growth performance in broilers is shown in Table 1 below, where supplementation of the diet with 0.055% oxazaborolidines increased the final body weight by 1.5%, decreased the overall FCR by 1.9% and increased the ADG in the Finisher phase by 2.4% compared to the basal dietary control.

TABLE 1

| | Control | Compound 5 |
|---|---|---|
| Initial BW, g | 41.3 | 41.3 |

Starter (1-7 d)

| | | |
|---|---|---|
| BW, g | 140 | 140.7 |
| ADG, g | 14.1 | 14.2 |
| ADFI, g | 15.4 | 15.5 |
| FCR | 1.09 | 1.08 |

Grower (8-21 d)

| | | |
|---|---|---|
| BW, g | 816.5 | 816.9 |
| ADG, g | 48.3 | 48.3 |
| ADFI, g | 68.5 | 68.3 |
| FCR | 1.42 | 1.41 |

Finisher (22-32 d)

| | | |
|---|---|---|
| BW, g | 2047.1 | 2078.6 |
| ADG, g | 87.9 | 90.1 |
| ADFI, g | 146.5 | 147.8 |
| FCR | 1.67 | 1.64 |

Overall (1-32 d)

| | | |
|---|---|---|
| ADG, g | 57.3 | 58.2 |
| ADFI, g | 89.1 | 89.5 |
| FCR | 1.55 | 1.52 |

### Example 5: Further Investigation into the Use of Pure Oxazaborolidine 2-butyl-3,4 dimethyl-5-phenyl 1,3,2-oxazaborolidine (Compound 2) and 3,4-dimethyl-2-5-diphenyl-1,3,2-oxazaborolidne (Compound 1) as a Growth Promotor in Poultry

The pure oxazaborolidines prepared by the method of Example 1 was investigated to assess its potential as a growth promotor in poultry.

1200 birds, 1-day old broiler (Ross 308; mixed sex) were treated over 32 days. 4 dietary groups were assessed with 6 replicates per diet; 50 birds per pen. The four Dietary groups consisted of (1) a control basal diet and (2) feed supplemented with a feed additive according to an embodiment of the present invention (0.1% 2-butyl-3,4 dimethyl-5-phenyl 1,3,2-oxazaborolidine-compound 2), (3) feed supplemented with a feed additive according to another embodiment of the present invention (0.1% 3,4-dimethyl-2-5-diphenyl-1,3,2-oxazaborolidine-compound 1) and (4) feed supplemented with the known antibiotic, ampicillin (0.1%).

The feeding programme followed a typical feeding program on a farm (3 phases: starter; grower; finisher) with the feed additives added to the basal diet with top dressing.

The following measurements were taken:

Growth Performance: Body weight (BW); Weight gain (Average Daily Gain—ADG); Feed intake (Average Daily Feed Intake—ADFI); FCR (food conversion ratio—feed input divided by feed output); Mortality Production Index.

Microbiome: Cecal samples were harvested at three time points (week 1, week 3 and week 5). Six replicates were harvested per dietary group. Total DNA was purified and the bacterial microbiome was analysed by 16S sequencing.

The effect of the additive on growth performance in broilers is shown in Table 2 below, where supplementation of the diet with 0.1% oxazaborolidines decreased the overall FCR by 0.59% and 1.76% for Compound 1 and Compound 2 respectively compared to the basal diet Control. Compound 2 showed an increase overall ADG and ADFI of 5.7% and 4.6% compared to the basal diet Control. Compound 2 showed an increase in body weight gain during all growing phases of 4.5%, 8.7% and 5.7%, corresponding to starter, grower and finisher phases respectively, compared to the basal diet Control. The results indicate that oxazaborolidines, particularly 2-butyl-3,4 dimethyl-5-phenyl 1,3,2-oxazaborolidine prepared according to the method of the present invention, improve growth and FCR in poultry.

TABLE 2

| | Treatment | | | | | |
|---|---|---|---|---|---|---|
| | CON | ATB | Compound 1 | Compound 2 | SEM² | P-value³ |
| Initial BW, g | 44.9 | 44.9 | 44.9 | 44.9 | 0.03 | 0.4962 |
| Starter (1-7 d) | | | | | | |
| BW, g | 173.9^{BC} | 199.1^{A} | 172.4^{C} | 182.0^{B} | 2.78 | 0.0001 |
| ADG, g | 18.4^{BC} | 22.0^{A} | 18.2^{C} | 19.6^{B} | 0.40 | 0.0001 |
| ADFI, g | 19.5^{B} | 21.9^{A} | 19.5^{B} | 20.4^{B} | 0.34 | 0.0004 |
| FCR | 1.06^{AB} | 0.99^{C} | 1.07^{A} | 1.04^{B} | 0.006 | 0.0001 |
| Mortality, % | 0.67 | 2.33 | 1.00 | 1.33 | 0.584 | 0.2496 |
| Grower (8-21 d) | | | | | | |
| BW, g | 915.0^{C} | 1074.6^{A} | 892.8^{C} | 1002.2^{B} | 19.32 | 0.0001 |
| ADG, g | 52.9^{C} | 62.6^{A} | 51.5^{C} | 58.6^{B} | 1.25 | 0.0001 |
| ADFI, g | 75.2^{C} | 85.9^{A} | 72.7^{C} | 80.7^{B} | 1.47 | 0.0001 |
| FCR | 1.47^{a} | 1.42^{bc} | 1.46^{ab} | 1.42^{c} | 0.013 | 0.0200 |
| Mortality, % | 1.83 | 1.53 | 0.37 | 1.12 | 0.905 | 0.6922 |
| Grower (22-32 d) | | | | | | |
| BW, g | 1734.1^{BC} | 1987.4^{A} | 1669.4^{C} | 1839.0^{B} | 32.26 | 0.0001 |
| ADG, g | 74.5^{BC} | 83.0^{A} | 70.6^{C} | 76.1^{B} | 1.68 | 0.0009 |
| ADFI, g | 140.0^{BC} | 155.9^{A} | 133.5^{C} | 144.0^{B} | 2.76 | 0.0003 |
| FCR | 2.06 | 2.06 | 2.08 | 2.09 | 0.031 | 0.8617 |
| Mortality, % | 0.42 | 0.00 | 0.40 | 0.42 | 0.379 | 0.8288 |
| Overall (1-32 d) | | | | | | |
| ADG, g | 52.8^{BC} | 60.7^{A} | 50.8^{C} | 56.1^{B} | 1.13 | 0.0001 |
| ADFI, g | 85.2^{BC} | 96.0^{A} | 82.0^{C} | 89.3^{B} | 1.59 | 0.0001 |
| FCR | 1.70^{x} | 1.66^{y} | 1.69^{x} | 1.67^{xy} | 0.012 | 0.0949 |
| Survivability, % | 96.7 | 95.4 | 97.9 | 96.7 | 1.21 | 0.5586 |

¹Values presented as least squares of the mean
²Standard error of the mean
³One-way ANOVA between all treatments
^{x,y}Means within a row with different superscripts tend to be different (P < 0.10).
^{a,b,c}Means with different superscripts in the same row significantly differ (P < 0.01)
^{A,B,C}Means with different superscripts in the same row significantly differ (P < 0.01)

The feed additives of the present invention are considered to act as antibacterial agents by acting on the gut flora (microbiome) of the birds, both against harmful gram-negative and gram-positive bacteria. While the effect of certain oxazaborolidine compounds on biofilms is known, different species have completely different flora within the gut and therefore it could not be predicted that such compounds would be effective in poultry.

This change of the bacterial population alters the distribution and make-up of a bird microbiome which may be manipulated to benefit overall gut health, Supplementation of poultry diets with Compound 1 and Compound 2 significantly increased the α-diversity of the cecal microbiome compared to the basal control in the first week (p=0.0184 and 0.0393 respectively; two-way ANOVA); as illustrated in FIG. 8 of the accompanying drawings which shows changes in bacterial α-diversity at the genus level, during the first, third and fifth week of dietary supplementation of broilers with oxazaborolidines. Plots display the smallest and largest value as the whiskers, with the box comprising the 25th percentile, median and 75th percentile. The supplementation of these oxazaborolidines into the diet significantly increases the diversity of the cecal microbiome at this early stage in the chicken development, which is typically associated with a more stable, and healthy, microbiome community.

1. A process for producing an oxazaborolidine, the process comprising the steps of:
(a) reacting an amino alcohol with a boronic acid with the azeotropic removal of water to provide a mixture of the starting materials and an oxazaborolidine end product; and
(b) purifying the oxazaborolidine end product to at least 75% purity.

2. The process according to claim 1 wherein the amino alcohol is selected from (−) ephedrine, (−) norpseudoephedrine, ethanolamine, ethanolamine, N-benzylethanolamine and a diol amine containing an aromatic moiety and the boronic acid is selected from a butyl- and phenylboronic acid.

3. (canceled)

4. (canceled)

5. (canceled)

6. The process according to claim 1, wherein purification step (b) comprises dissolving a bicarboxylic acid in water and mixing this solution with the product of step (a) to precipitate out the starting material as a salt and produce an oxazaborolidine product filtrate.

7. The process according to claim 6 wherein the bicarboxylic acid is selected from oxalic, adipic and citric acid.

8. The process according to claim 6, further comprising heating the bicarboxylic acid in water in step (b) to at least 40° C., filtering the precipitate and drying the product filtrate by evaporation.

9. The process according to claim 1 wherein purification step (b) comprises a vacuum distillation step to provide substantially pure oxazaborolidine.

10. The process according to claim 9 wherein any organic solvent with the impure oxazaborolidine prepared in step (a) is removed and the residue mixed with a solvent, and subjected to vacuum distillation.

11. The process according to claim 9 wherein any organic solvent with the impure oxazaborolidine prepared in step (a) is removed and the residue mixed with a solvent, and subjected to vacuum distillation wherein the solvent is dichloromethane (DCM).

12. The process according to claim 9 wherein distillation is carried out at least 0.5 mbar and at least 100° C., to provide a substantially pure oxazaborolidine as the distillate.

13. The process according to claim 9 wherein Kugelrohr vacuum distillation is used to obtain the pure product.

14. The process according to claim 9 wherein the amino alcohol in step (a) is selected from ethanolamine and N-benzyl ethanolamine and the boronic acid in step (a) is phenylboronic acid.

15. A process for producing an oxazaborolidine, the process comprising the steps of:
(a) reacting an amino alcohol with trihydridoboron (BH₃) in an organic solvent; and
(b) immediately alkylating an unalkylated intermediate compound produced by step (a) using Alkyl Lithium to provide the oxazaborolidine end product.

16. The process according to claim 15, wherein the organic solvent used in step (a) is selected from DMS and THF.

17. The process according to claim 15 wherein step (a) is carried out at a temperature of at least 60° C.

18. The process according to claim 15, wherein the alkyl lithium in step (b) is selected from Butyl- or Phenyl Lithium.

19. The process according to of claim 15, wherein step (b) is carried out at a temperature of −50° C.

20. A substantially pure oxazaborolidine having the general formula (I) below prepared according to the process of claim 1: 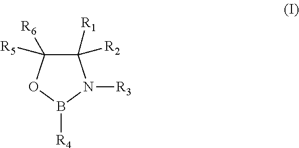
wherein
R1, R2, and R3 are each independently selected from hydrogen, C₁-C₈ alkyl, C₂-C₈ alkenyl, aryl, and C₃-C₇ cycloalkyl or one of R1 and R2 together with R3 form a substituted or unsubstituted aromatic ring, a substituted or unsubstituted cycloalkyl ring, or a substituted or unsubstituted heterocyclic ring, fused to the oxazaborolidine ring;
R4 is selected from hydrogen, hydroxyl, C₁-C₈ alkyl, C₂-C₈ alkenyl, aryl, and C₃-C₇ cycloalkyl;
R5 and R6 are each independently selected from hydrogen, C₁-C₈ alkyl, C₂-C₈ alkenyl, aryl, and C₃-C₇ cycloalkyl.

21. A feed additive for an animal or bird, especially poultry, wherein the feed additive comprises an oxazaborolidine according to claim 20.

22. A feed additive for an animal or bird, especially poultry, wherein the feed additive comprises an oxazaborolidine according to claim 20 added to a basal diet in a substantially pure form in a dose of 100-300 g per tonne of animal feed.

23. The feed additive as claimed in claim 21 comprising the oxazaborolidine, 2-butyl-3,4 dimethyl-5-phenyl 1,3,2-oxazaborolidine.
